(19) United States
(12) Patent Application Publication
(10) Pub. No.: US 2026/0199370 A1
(43) Pub. Date: Jul. 16, 2026

(54) COCRYSTALS OF ODEVIXIBAT

(71) Applicant: ALBIREO AB, Göteborg (SE)

(72) Inventors: I. Fraser PICKERSGILL, Newtown, PA (US); Praveen K. SHARMA, Arlington, MA (US); Benjamin KOHN, Somerville, MA (US)

(21) Appl. No.: 19/137,175

(22) PCT Filed: Dec. 11, 2023
(86) PCT No.: PCT/EP2023/085128

§ 371 Date: Jun. 9, 2025

(30) Foreign Application Priority Data
Dec. 9, 2022 (SE) 2230407-5

(51) Int. Cl. A61K 31/554; A61K 47/22

(52) U.S. Cl. CPC A61K 31/554; A61K 47/22

(57) ABSTRACT

The present invention relates to a cocrystal of 1,1-dioxo-3,3-dibutyl-5-phenyl-7-methylthio-8-(N-{(R)-α-[N-((S)-1-carboxypropyl) carbamoyl]-4-hydroxybenzyl}carbamoylmethoxy)-2,3,4,5-tetrahydro-1,2,5-benzothiadiazepine (odevixibat) and pyridoxine. The invention also relates to a pharmaceutical composition comprising said cocrystal, and to its use in the treatment of various conditions as described herein.

The present invention relates to a cocrystal of 1,1-dioxo-3,3-dibutyl-5-phenyl-7-methylthio-8-(N-{(R)-α-[N-((S)-1-carboxypropyl) carbamoyl]-4-hydroxybenzyl}carbamoylmethoxy)-2,3,4,5-tetrahydro-1,2,5-benzothiadiazepine (odevixibat) and pyridoxine with high chemical and physical stability. The invention also relates to a pharmaceutical composition comprising said cocrystal, and to its use in the treatment of various conditions as described herein.

### BACKGROUND

WO 03/022286 discloses the compound 1,1-dioxo-3,3-dibutyl-5-phenyl-7-methylthio-8-(N-{(R)-α-[N-((S)-1-carboxypropyl) carbamoyl]-4-hydroxybenzyl}carbamoylmethoxy)-2,3,4,5-tetrahydro-1,2,5-benzothiadiazepine (odevixibat). As an ileal bile acid transporter (IBAT) inhibitor, odevixibat prevents the natural reabsorption of bile acids from the ileum into the hepatic portal circulation, leading to unabsorbed bile acids being excreted via faeces. This results in reduced levels of bile acids in the serum and in the liver. Odevixibat is therefore useful in the treatment or prevention of diseases such as dyslipidemia, constipation, diabetes and liver diseases, and especially liver diseases that are associated with elevated bile acid levels.

According to the experimental section of WO 03/022286, the last step in the preparation of odevixibat involves the hydrolysis of a tert-butyl ester under acidic conditions. The crude compound was obtained by evaporation of the solvent under reduced pressure followed by purification of the residue by preparative HPLC (Example 29). No crystalline material was identified. Amorphous materials may contain residual solvents, which is highly undesirable for materials aimed for pharmaceutical use. Also, because of their lower chemical and physical stability, as compared with crystalline material, amorphous materials may display faster decomposition and may spontaneously form crystals with a variable degree of crystallinity. This may result in unreproducible solubility rates and difficulties in storing and handling the material. In pharmaceutical preparations, the active pharmaceutical ingredient (API) is for that reason preferably used in a highly crystalline state.

WO 2019/245448 discloses a crystalline sesquihydrate of odevixibat, and its preparation from certain solvents or solvent mixtures.

Notwithstanding, there is a need for additional crystal forms of odevixibat having improved properties with respect to stability, bulk handling and solubility. It is therefore an object of the present invention to provide a stable crystal form of odevixibat with good crystallinity and good formulation properties.

### BRIEF DESCRIPTION OF THE DRAWINGS

FIG. 1 shows the X-ray powder diffractogram of the cocrystal of odevixibat and pyridoxine.

FIG. 2 shows the DSC thermogram of the cocrystal.

FIG. 3 shows the TG-FTIR thermogram of the cocrystal.

FIG. 4 shows the DVS isotherm of the cocrystal, with the change of water content as a function of time.

FIG. 5 shows the DVS isotherm of the cocrystal, with the change of water content as a function of relative humidity.

FIG. 6 shows overlaying X-ray powder diffractograms of the cocrystal before (bottom) and after (top) dynamic vapor sorption experiments.

### DETAILED DESCRIPTION OF THE INVENTION

It has been discovered that stable crystalline forms of odevixibat can be obtained by crystallization of odevixibat together with certain cocrystal formers. In a first aspect, therefore, the invention relates to a cocrystal of odevixibat and pyridoxine. As described in the experimental section, the cocrystal can be obtained e.g. from an isopropanol solution of odevixibat and pyridoxine. It has been determined by ¹H NMR that the cocrystal contains odevixibat and pyridoxine in a 2:1 molar ratio (data not shown). In one embodiment, the invention relates to a cocrystal of odevixibat and pyridoxine, wherein odevixibat and pyridoxine are present in a 2:1 molar ratio.

It has been observed that the cocrystal retains water in a manner that is reversable and continuous. At 95% relative humidity, approximately 2.5% water is retained. By reducing the humidity to zero, the cocrystal loses all its water. A monohydrate would have an expected water content of 2.1%, thus it is believed that the cocrystal is a non-stoichiometric hydrate that contains typically about 1% water under ambient storage conditions.

In some embodiments, the invention relates to a cocrystal of odevixibat and pyridoxine having an X-ray powder diffraction (XRPD) pattern, obtained with CuKα1-radiation, with at least three specific peaks at ° 2θ positions selected from the list consisting of 4.70±0.2, 6.13±0.2, 8.02±0.2, 9.22±0.2, 11.32±0.2, 11.52±0.2, 12.92±0.2, 15.93±0.2, 16.76±0.2 and 24.12±0.2.

In some embodiments, the invention relates to a cocrystal of odevixibat and pyridoxine having an XRPD pattern, obtained with CuKα1-radiation, with at least three specific peaks at ° 2θ positions selected from the list consisting of 4.70±0.2, 6.13±0.2, 8.02±0.2, and 11.52±0.2.

In some embodiments, the invention relates to a cocrystal of odevixibat and pyridoxine having an XRPD pattern, obtained with CuKα1-radiation, with specific peaks at ° 2θ positions 4.70±0.2, 6.13±0.2, 8.02±0.2, and 11.52±0.2 and with one or more of 12.92±0.2, 15.93±0.2, and 16.76±0.2.

In some embodiments, the invention relates to a cocrystal of odevixibat and pyridoxine having an XRPD pattern, obtained with CuKα1-radiation, with specific peaks at ° 2θ positions 4.70±0.2, 6.13±0.2, 8.02±0.2, 11.52±0.2, 12.92±0.2, and 16.76±0.2.

In some embodiments, the cocrystal of odevixibat and pyridoxine has an XRPD pattern, obtained with CuKα1-radiation, with specific peaks at ° 2θ positions 4.70±0.2, 6.13±0.2, 8.02±0.2, 9.22±0.2, 11.32±0.2, 11.52±0.2, 12.92±0.2, 15.93±0.2, 16.76±0.2 and 24.12±0.2.

In some embodiments, the cocrystal of odevixibat and pyridoxine has an XRPD pattern, obtained with CuKα1-radiation, with specific peaks at ° 2θ positions 4.70±0.2, 6.13±0.2, 8.02±0.2, 9.22±0.2, 11.32±0.2, 11.52±0.2, 12.92±0.2, 15.93±0.2, 16.76±0.2 and 24.15±0.2, and one or more of 8.28±0.2, 10.17±0.2, 13.32±0.2, 14.07±0.2, 14.62±0.2, 16.44±0.2, 18.13±0.2, 18.73±0.2, 19.29±0.2, 24.48±0.2 and 25.45±0.2.

In some embodiments, the cocrystal of odevixibat and pyridoxine has an XRPD pattern, obtained with CuKα1-radiation, with specific peaks at ° 2θ positions 4.70±0.2, 6.13±0.2, 8.02±0.2, 8.28±0.2, 9.22±0.2, 10.17±0.2, 11.32±0.2, 11.52±0.2, 12.92±0.2, 13.32±0.2, 14.07±0.2, 14.62±0.2, 15.93±0.2, 16.44±0.2, 16.76±0.2, 18.13±0.2, 18.73±0.2, 19.29±0.2, 24.15±0.2, 24.48±0.2 and 25.45±0.2.

In some embodiments, the invention relates to a cocrystal of odevixibat and pyridoxine having a XRPD pattern, obtained with CuKα1-radiation, with specific peaks as disclosed in Table 1.

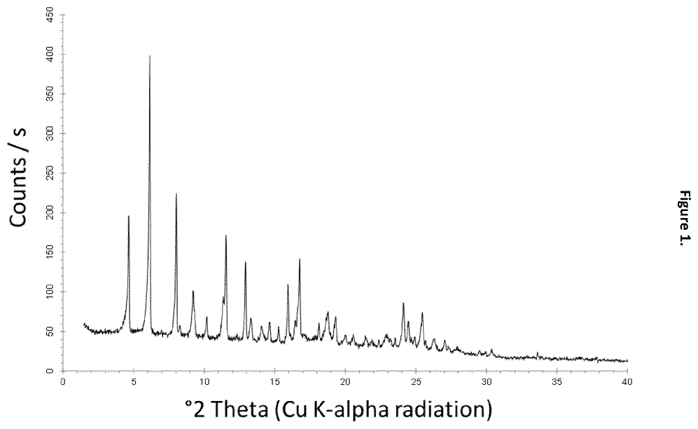

In some embodiments, the invention relates to a cocrystal of odevixibat and pyridoxine having a XRPD pattern, obtained with CuKα1-radiation, substantially as shown in FIG. 1.

In some embodiments, the invention relates to a cocrystal of odevixibat and pyridoxine, wherein the DSC curve of the cocrystal comprises an endotherm between about 143° C. and about 160° C., such as at approximately 145.7° C.

In some embodiments, the invention relates to a cocrystal of odevixibat and pyridoxine, wherein the crystallinity is greater than 99%

In a second aspect, the invention relates to a pharmaceutical composition comprising a therapeutically effective amount of the cocrystal, as disclosed herein, together with one or more pharmaceutically acceptable excipients. Excipients may include fillers, binders, surfactants, disintegrants, glidants and lubricants. The pharmaceutical composition may further comprise at least one other active substance, such as an active substance selected from an IBAT inhibitor; an enteroendocrine peptide or enhancer thereof; a dipeptidyl peptidase-IV inhibitor; a biguanidine; an incretin mimetic; a thiazolidinone; a PPAR agonist; a HMG Co-A reductase inhibitor; a bile acid binder; a TGR5 receptor modulator; a member of the prostone class of compounds; a guanylate cyclase C agonist; a 5-HT4 serotonin agonist; or a pharmaceutically acceptable salt of any one these active substances. Examples of such combinations are disclosed in WO2012/064268.

In one embodiment, the invention relates to the pharmaceutical composition as disclosed herein, wherein the polymorphic purity of the cocrystal is at least about 90%. In some embodiments, the polymorphic purity is at least about 95%. In some embodiments, the polymorphic purity is at least about 98%. For example, the polymorphic purity may be at least about 98.5%, such as at least about 99%, such as at least about 99.5%, such as at least about 99.8%, or such as at least about 99.9%.

In general, pharmaceutical compositions may be prepared in a conventional manner using conventional excipients. In some embodiments, the ingredients of the formulation are mixed to a homogenous mixture and then formulated as tablets or capsules. The homogenous mixture of the ingredients may be compressed into tablets using conventional techniques, such as the rotary tablet press technique. The mixture of ingredients may also be granulated. For instance, the mixture of ingredients may be wetted by the addition of a liquid, such as water and/or an appropriate organic solvent (e.g., ethanol or isopropanol), and thereafter granulated and dried. Alternatively, granules may be prepared by dry granulation, such as by roller compaction. The granules obtained may be compressed into tablets using conventional techniques. Capsules may comprise a powder mixture or small multiparticulates (such as granules, extruded pellets or minitablets) of the ingredients. If desirable, any of the tablets, capsules, granules, extruded pellets and minitablets mentioned above may be coated with one or more coating layers. Such coating layers may be applied by methods known in the art, such as by film coating involving perforated pans and fluidized beds. In some embodiments, the formulation is in the form of a tablet.

In a yet further aspect, the invention relates to the cocrystal of odevixibat and pyridoxine, as disclosed herein, for use in therapy.

Odevixibat is an ileal bile acid transporter (IBAT) inhibitor. The ileal bile acid transporter (IBAT) is the main mechanism for re-absorption of bile acids from the GI tract. Partial or full blockade of that IBAT mechanism will result in lower concentration of bile acids in the small bowel wall, portal vein, liver parenchyma, intrahepatic biliary tree, and extrahepatic biliary tree, including the gall bladder. Diseases which may result from partial or full blockade of the IBAT mechanism may be those having, as a primary pathophysiological defect, symptoms of excessive concentration of bile acids in serum and in the above organs. The cocrystal, as described herein, is therefore useful in the treatment or prevention of conditions, disorders and diseases wherein inhibition of the bile acid circulation is desirable, such as cardiovascular diseases or disorders, fatty acid metabolism and glucose utilization disorders, gastrointestinal diseases and disorders, liver diseases and disorders, and hyperabsorption syndromes.

Cardiovascular disorders or diseases, or a disorder of fatty acid metabolism, or a glucose utilization disorder include hypercholesterolemia; disorders in which insulin resistance is involved; disorders of fatty acid metabolism; type 1 and type 2 diabetes mellitus; complications of diabetes, including cataracts, micro- and macrovascular diseases, retinopathy, neuropathy, nephropathy and delayed wound healing, tissue ischaemia, diabetic foot; arteriosclerosis; myocardial infarction; acute coronary syndrome; unstable angina pectoris; stable angina pectoris; stroke; peripheral arterial occlusive disease; cardiomyopathy; heart failure; heart rhythm disorders and vascular restenosis; diabetes-related diseases such as insulin resistance (impaired glucose homeostasis), hyperglycemia, hyperinsulinemia, elevated blood levels of fatty acids or glycerol, obesity, dyslipidemia, hyperlipidemia including hypertriglyceridemia, metabolic syndrome (syndrome X), atherosclerosis and hypertension; and increased high density lipoprotein levels.

Gastrointestinal diseases or disorders include constipation; chronic constipation; functional constipation; chronic idiopathic constipation (CIC); intermittent/sporadic constipation; constipation secondary to diabetes mellitus; constipation secondary to stroke; constipation secondary to chronic kidney disease; constipation secondary to multiple sclerosis; constipation secondary to Parkinson's disease; constipation secondary to systemic sclerosis; drug induced constipation; irritable bowel syndrome with constipation (IBS-C); irritable bowel syndrome mixed (IBS-M); pediatric functional constipation and opioid induced constipation; Crohn's disease; primary bile acid malabsorption; irritable bowel syndrome (IBS); inflammatory bowel disease (IBD); ileal inflammation; and reflux disease and complications thereof, such as Barrett's esophagus, bile reflux esophagitis and bile reflux gastritis.

A liver disease or disorder is defined herein as an inherited metabolic disorder of the liver; inborn errors of bile acid synthesis; congenital bile duct anomalies; biliary atresia; post-Kasai biliary atresia; post-liver transplantation biliary atresia; neonatal hepatitis; neonatal cholestasis; hereditary forms of cholestasis; cerebrotendinous xanthomatosis; a secondary defect of bile acid (BA) synthesis; Zellweger's syndrome; cystic fibrosis-associated liver disease; alpha1-antitrypsin deficiency; Alagilles syndrome (ALGS); Byler syndrome; a primary defect of bile acid (BA) synthesis; progressive familial intrahepatic cholestasis (PFIC) including PFIC-1, PFIC-2, PFIC-3 and non-specified PFIC, post-biliary diversion PFIC and post-liver transplant PFIC; benign recurrent intrahepatic cholestasis (BRIC) including BRIC1, BRIC2 and non-specified BRIC, post-biliary diversion BRIC and post-liver transplant BRIC; autoimmune hepatitis; primary biliary cirrhosis (PBC); liver fibrosis; non-alcoholic fatty liver disease (NAFLD); non-alcoholic steatohepatitis (NASH); portal hypertension; cholestasis; Down syndrome cholestasis; drug-induced cholestasis; intrahepatic cholestasis of pregnancy; jaundice during pregnancy; intrahepatic cholestasis; extrahepatic cholestasis; parenteral nutrition associated cholestasis (PNAC); low phospholipid-associated cholestasis; lymphedema cholestasis syndrome 1 (LSC1); primary sclerosing cholangitis (PSC); immunoglobulin G4 associated cholangitis; primary biliary cholangitis; cholelithiasis (gall stones); biliary lithiasis; choledocholithiasis; gallstone pancreatitis; Caroli disease; malignancy of bile ducts; malignancy causing obstruction of the biliary tree; biliary strictures; AIDS cholangiopathy; ischemic cholangiopathy; pruritus due to cholestasis or jaundice; pancreatitis; chronic autoimmune liver disease leading to progressive cholestasis; hepatic steatosis; alcoholic hepatitis; acute fatty liver; fatty liver of pregnancy; drug-induced hepatitis; iron overload disorders; congenital bile acid synthesis defect type 1 (BAS type 1); drug-induced liver injury (DILI); hepatic fibrosis; congenital hepatic fibrosis; hepatic cirrhosis; Langerhans cell histiocytosis (LCH); neonatal ichthyosis sclerosing cholangitis (NISCH); erythropoietic protoporphyria (EPP); idiopathic adulthood ductopenia (IAD); idiopathic neonatal hepatitis (INH); non syndromic paucity of interlobular bile ducts (NS PILBD); North American Indian childhood cirrhosis (NAIC); hepatic sarcoidosis; amyloidosis; necrotizing enterocolitis; serum bile acid-caused toxicities, including cardiac rhythm disturbances (e.g., atrial fibrillation) in setting of abnormal serum bile acid profile, cardiomyopathy associated with liver cirrhosis (“cholecardia”), and skeletal muscle wasting associated with cholestatic liver disease; viral hepatitis (including hepatitis A, hepatitis B, hepatitis C, hepatitis D and hepatitis E); hepatocellular carcinoma (hepatoma); cholangiocarcinoma; bile acid-related gastrointestinal cancers; and cholestasis caused by tumours and neoplasms of the liver, of the biliary tract and of the pancreas. The cocrystal can also be used in the enhancement of corticosteroid therapy in liver disease.

Hyperabsorption syndromes include abetalipoproteinemia, familial hypobetalipoproteinemia (FHBL), chylomicron retention disease (CRD) and sitosterolemia; hypervitaminosis and osteopetrosis; hypertension; glomerular hyperfiltration; and pruritus of renal failure. The cocrystal can also be used in the protection against liver- or metabolic disease-associated kidney injury.

In one embodiment, the invention relates to a cocrystal of odevixibat and pyridoxine, as described herein, for use in the treatment or prevention of a disease or disorder as listed above.

In another embodiment, the invention relates to the use of a cocrystal of odevixibat and pyridoxine, as described herein, in the manufacture of a medicament for the treatment or prevention of a disease or disorder as listed above.

Crystalline forms of odevixibat will normally be administered to a warm-blooded animal at a unit dose within the range of about 0.01 to about 1.0 mg/kg, such as about 0.01 to about 0.5 mg/kg, or such as about 0.01 to about 0.2 mg/kg, and this normally provides a therapeutically effective dose. A unit dose form, such as a tablet or capsule, will usually contain about 0.1 to about 20 mg of active ingredient, such as about 0.1 to about 10 mg, or such as about 0.2 to about 5 mg, or such as about 0.2 to about 1.0 mg. For example, the unit dose form may contain about 0.2 mg, about 0.4 mg, about 0.6 mg, or about 1.2 mg of active ingredient. The daily dose can be administered as a single dose or divided into two, three or more unit doses. An orally administered daily dose of an IBAT inhibitor is preferably within about 0.1 to about 50 mg, more preferably within about 0.1 to about 20 mg, such as within about 0.2 to about 10 mg, or such as within about 0.2 to about 5.0 mg.

The dosage required for the therapeutic or prophylactic treatment will depend on the route of administration, the severity of the disease, the age and weight of the patient and other factors normally considered by the attending physician when determining the individual regimen and dosage levels appropriate for a particular patient.

### Definitions

As used herein, the term “cocrystal” refers to a crystalline form (or polymorph) comprised of at least two components, such as at least two molecules.

As used herein, the term “polymorph” refers to crystals of the same molecule (or the same combination of at least two molecules) that have different physical properties as a result of the order of the molecules in the crystal lattice. Polymorphs of a single compound have one or more different chemical, physical, mechanical, electrical, thermodynamic, and/or biological properties from each other. Differences in physical properties exhibited by polymorphs can affect pharmaceutical parameters such as storage stability, compressibility, density (important in composition and product manufacturing), dissolution rates (an important factor in determining bioavailability), solubility, melting point, chemical stability, physical stability, powder flowability, water sorption, compaction, and particle morphology. Differences in stability can result from changes in chemical reactivity (e.g. differential oxidation, such that a dosage form discolours more rapidly when comprised of one polymorph than when comprised of another polymorph) or mechanical changes (e.g., crystal changes on storage as a kinetically favoured polymorph converts to a thermodynamically more stable polymorph) or both (e.g., one polymorph is more hygroscopic than the other). As a result of solubility/dissolution differences, some transitions affect potency and/or toxicity. In addition, the physical properties of the crystal may be important in processing; for example, one polymorph might be more likely to form solvates or might be difficult to filter and wash free of impurities (i.e., particle shape and size distribution might be different between one polymorph relative to the other). “Polymorph” does not include amorphous forms of the compound.

As used herein, the terms “treatment,” “treat,” and “treating” refer to reversing, alleviating, delaying the onset of, or inhibiting the progress of a disease or disorder, or one or more symptoms thereof, as described herein. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

As used herein, the term “pharmaceutically acceptable” refers to those compounds, materials, compositions and/or dosage forms that are suitable for human pharmaceutical use and that are generally safe, non-toxic and neither biologically nor otherwise undesirable.

As used herein, the term “about” refers to a value or parameter herein that includes (and describes) embodiments that are directed to that value or parameter per se. For example, description referring to “about 20” includes description of “20.” Numeric ranges are inclusive of the numbers defining the range. Generally speaking, the term “about” refers to the indicated value of the variable and to all values of the variable that are within the experimental error of the indicated value (e.g., within the 95% confidence interval for the mean) or within 10 percent of the indicated value, whichever is greater.

The crystallinity of the cocrystal of odevixibat and pyridoxine may be measured e.g. by X-ray powder diffraction (XRPD) methods or by differential scanning calorimetry (DSC) methods. When reference is made herein to a crystalline compound, preferably the crystallinity is greater than about 70%, such as greater than about 80%, particularly greater than about 90%, more particularly greater than about 95%. In some embodiments of the invention, the degree of crystallinity is greater than about 98%, preferably greater than about 99%. In one embodiment, the crystallinity is between about 0 and about 100%, preferably between about 50 and about 100%, more preferably between about 90 and about 100%, and even more preferably between about 95 and about 100%. The % crystallinity refers to the percentage by weight of the total sample mass which is crystalline.

As used herein, the term “polymorphic purity” when used in reference to a composition comprising a polymorph of odevixibat, refers to the percentage of one specific polymorph relative to another polymorph or an amorphous form of odevixibat in the referenced composition. For example, a composition comprising a cocrystal of odevixibat and pyridoxine having a polymorphic purity of 90% would comprise 90 weight parts of the cocrystal and 10 weight parts of other crystalline and/or amorphous forms of odevixibat. Preferably, the described cocrystal of odevixibat and pyridoxine includes less than, for example, 20%, 15%, 10%, 5%, 3%, or particularly, less than 1% by weight of other polymorphs of odevixibat. Thus, preferably, the polymorphic purity of the described cocrystal of odevixibat is >80%, >85%, >90%, >95%, >97%, or particularly >99%.

The below examples describing the invention do not limit the invention in any respect. All cited documents and references are incorporated by reference.

### ABBREVIATIONS

DMSO dimethylformamide
TMS trimethylsilane
RH relative humidity
NMR nuclear magnetic resonance
equiv equivalent

### General Experimental Methods

### Powder X-Ray Diffraction (XRPD) Analysis

These analyses were carried out with a Stoe Stadi P diffractometer equipped with a Mythen1K detector operating with Cu-Kal radiation. A curved Ge monochromator allows testing with Cu-Kal radiation. For a typical sample preparation about 10 mg of sample was placed between two acetate foils and mounted into a Stoe transmission sample holder.

The samples were spun during analysis in order to increase the randomness of the samples. The following experimental settings were used:
Ambient air atmosphere
Tube voltage and power: 40 kV, 40 mA
0.02° 2θ step size
12 s step time
1.5-50.5° 2θ scanning range
1°2θ detector step

It is known in the art that an X-ray powder diffraction pattern may be obtained having one or more measurement errors depending on the measurement conditions (such as equipment, sample preparation or machine used). In particular, it is generally known that intensities in an XRPD pattern may fluctuate depending on measurement conditions and sample preparation. For example, persons skilled in the art of XRPD will realise that the relative intensities of peaks may vary according to the orientation of the sample under the test and on the type and setting of the instrument used. The skilled person will also realise that the position of reflections can be affected by the precise height at which the sample sits in the diffractometer and the zero calibration of the diffractometer. The surface planarity of the sample may also have a small effect. Hence a person skilled in the art will appreciate that the diffraction pattern presented herein is not to be construed as absolute and any crystalline form that provides a powder diffraction pattern substantially identical to those disclosed herein fall within the scope of the present disclosure (for further information, see R. Jenkins and R. L. Snyder, “Introduction to X-ray powder diffractometry”, John Wiley & Sons, 1996).

### Thermogravimetry Coupled to Fourier Transform Infrared Spectroscopy (TG-FTIR)

Thermogravimetric measurements were carried out with a Netzsch Thermo-Microbalance TG 209 coupled to a Bruker FTIR Spectrometer Vector 22 using sample pans with a pinhole, N₂ atmosphere, and a heating rate of 10° C./min up to 300° C.

### Dynamic Vapour Sorption (DVS)

DVS measurements were performed with an SPS11-100n “Sorptions Prüfsystem” from ProUmid (formerly “Projekt Messtechnik”), August-Nagel-Str. 23, 89079 Ulm (Germany).

About 5-20 mg of sample was put into an aluminum sample pan. Humidity change rates of 5% per hour were used.

The sample was placed on an aluminum or platinum holder on top of a microbalance and allowed to equilibrate at 50% RH before starting the pre-defined humidity programs:

$$2 h at 50 \% RH \qquad ( 1 )$$

$$50 \to 0 \% RH ( 5 \% / h ) ; 5 h at 0 \% RH \qquad ( 2 )$$

$$0 \to 95 \% RH ( 5 \% / h ) ; 5 h at 95 \% RH \qquad ( 3 )$$

$$95 \to 0 \% RH ( 5 \% / h ) ; 5 h at 0 \% RH \qquad ( 4 )$$

$$0 \to 95 \% RH ( 5 \% / h ) ; 5 h at 95 \% RH \qquad ( 5 )$$

$$95 \to 50 \% RH ( 5 \% / h ) ; 2 h at 50 \% RH \qquad ( 6 )$$

### Differential Scanning Calorimetry (DSC)

DSC measurements were performed with a TA Q2000 instrument (closed aluminum sample pan with or without a pinhole in the lid, heating rate 10° C./min). The melting point is understood as the peak maximum.

### EXAMPLES

### Example 1

Preparation of odevixibat/pyridoxine co-crystal

515.9 mg of odevixibat (0.67 mmol) was dissolved in 5 ml of 1-propanol at 40° C. 113.7 mg of pyridoxine (1 equiv.) was added to the clear solution at 40° C. Heating was stopped and the temperature was decreased to room temperature and the mixture was stirred for one day. After shaking, a cloudy solution was obtained, and a fine suspension formed. After one additional day of stirring, a suspension was obtained. Then, the vial was opened to allow solvent evaporation and stirring was continued for 5 further days. Light microscopy then revealed crystalline material and the suspension was filtered over a porosity 4 fritted glass filter. The filter cake was dried in air on the filter for 40 minutes and then submitted for XPRD (Table 1, FIG. 1). 284 mg of powder was recovered. ¹H NMR data showed that the crystalline material consists of odevixibat and pyridoxine in a 2:1 molar ratio (data not shown).

TABLE 1
XRPD peaks for the cocrystal

| Position [°2- | | |
|---|---|---|
| theta] | d-spacing [Å] | Rel. Int. [%] |
| 4.7 | 19.0 | 54 |
| 6.13 | 14.42 | 100 |
| 8.02 | 11.02 | 61 |
| 8.28 | 10.67 | 16 |
| 9.22 | 9.58 | 28 |
| 10.17 | 8.69 | 18 |
| 11.32 | 7.81 | 24 |
| 11.52 | 7.68 | 47 |
| 12.92 | 6.85 | 38 |
| 13.32 | 6.64 | 18 |
| 14.07 | 6.29 | 16 |
| 14.62 | 6.05 | 17 |
| 15.27 | 5.80 | 15 |
| 15.93 | 5.56 | 30 |
| 16.44 | 5.39 | 18 |
| 16.76 | 5.29 | 39 |
| 17.08 | 5.19 | 13 |
| 18.13 | 4.89 | 17 |
| 18.73 | 4.74 | 20 |
| 19.29 | 4.60 | 18 |
| 19.99 | 4.44 | 12 |
| 20.56 | 4.32 | 13 |
| 21.47 | 4.14 | 12 |
| 22.38 | 3.97 | 11 |
| 22.91 | 3.88 | 12 |
| 23.19 | 3.83 | 11 |
| 23.53 | 3.78 | 12 |
| 24.12 | 3.69 | 24 |
| 24.48 | 3.63 | 17 |
| 24.91 | 3.57 | 12 |
| 25.45 | 3.50 | 20 |
| 26.28 | 3.39 | 11 |
| 27.06 | 3.29 | 11 |
| 27.33 | 3.26 | 8 |
| 29.50 | 3.03 | 7 |
| 29.92 | 2.98 | 7 |
| 30.36 | 2.94 | 8 |
| 33.63 | 2.66 | 7 |

* The relative intensity depends on the particle orientation, crystallite size/shape, strain and specimen thickness

### Differential Scanning Calorimetry (DSC) Analysis

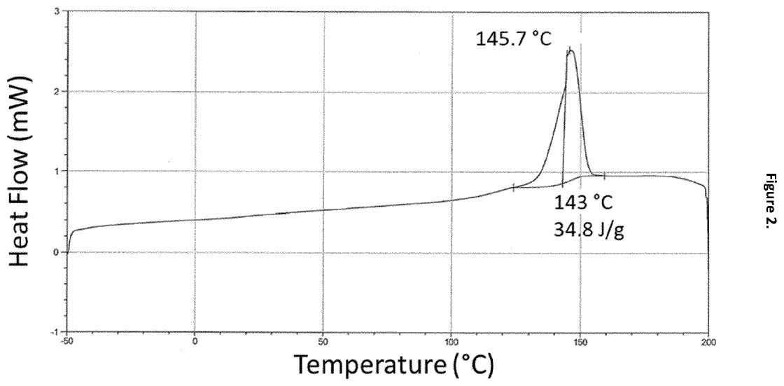

The melting point was measured to 145.7° C. with an onset at 143° C., as shown in FIG. 2.

### Thermogravimetric Analysis

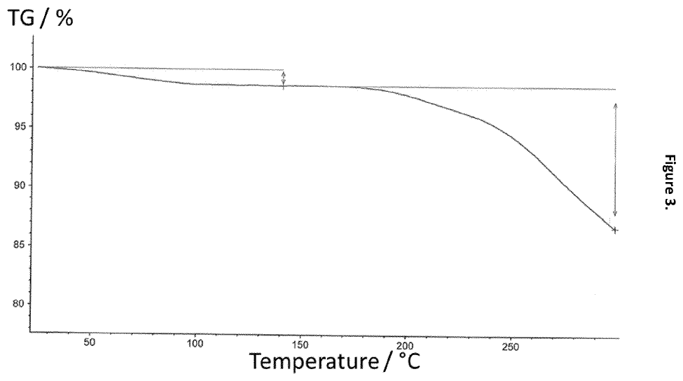

Thermogravimetric analysis showed a water loss of 1.4% between 25° C. and 140° C., corresponding to the evaporation of surface water. Decomposition was observed above 170° C., as shown in the TG-FTIR thermogram in FIG. 3.

### Dynamic Vapour Sorption (DVS) Analysis

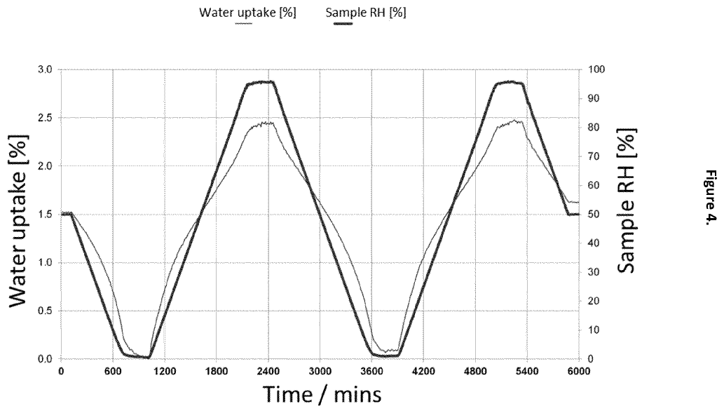

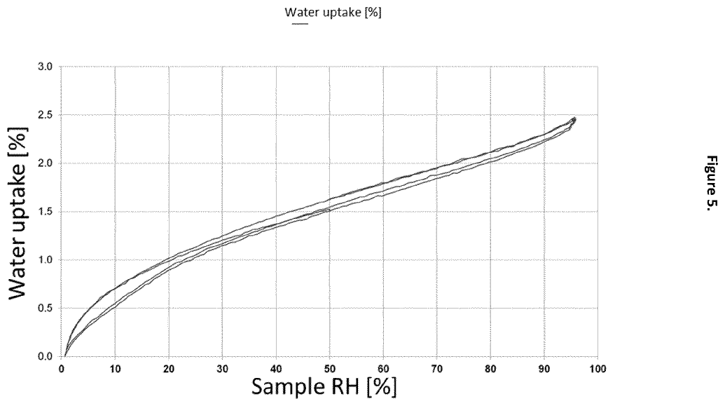

The behaviour of the cocrystal was investigated in presence of variable water vapour pressure using DVS measurement. At the initiation of the measurement, the sample contained approximately 1.5% of water at 50% relative humidity; this observation is consistent with the TG-FTIR result. When decreasing the relative humidity to 0%, the sample lost all its water. Upon storage at 95% relative humidity, the sample absorbed almost 2.5% of water. The result from the DVS test is presented in FIGS. 4 and 5.

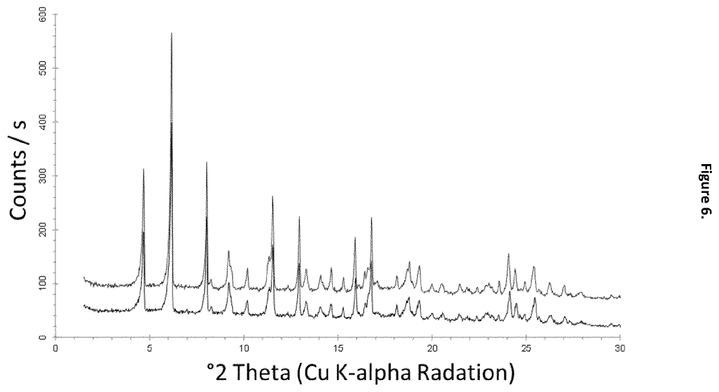

The water sorption appears to be reversible and continuous. As a monohydrate would have an expected water content of 2.1%, it is believed that the pyridoxine cocrystal is a non-stoichiometric hydrate that contains typically about 1% water under ambient storage conditions, but the water content can vary from 0 to about 2.5%. After the DVS test, the sample was recovered and submitted to XPRD and no change in the solid form was observed (FIG. 6).

1. A cocrystal of odevixibat and pyridoxine.

2. The cocrystal according to claim 1, wherein the odevixibat and the pyridoxine are present in a 2:1 molar ratio.

3. The cocrystal according to claim 1, having an XRPD pattern, obtained with CuKα1-radiation, with at least three specific peaks at °2θ positions selected from the list consisting of 4.70±0.2, 6.13±0.2, 8.02±0.2, 9.22±0.2, 11.32±0.2, 11.52±0.2, 12.92±0.2, 15.93±0.2, 16.76±0.2 and 24.12±0.2.

4. The cocrystal according to any one of the previous claim 1, having an XRPD pattern, obtained with CuKα1-radiation, with at least three specific peaks at °20 positions selected from the list consisting of 4.70±0.2, 6.13±0.2, 8.02±0.2, and 11.52±0.2.

5. The cocrystal according to any one of the previous claim 1, having an XRPD pattern, obtained with CuKα1-radiation, with specific peaks at °20 positions 4.70±0.2, 6.13±0.2, 8.02±0.2, and 11.52±0.2, and with one or more additional specific peaks at 12.92±0.2, 15.93±0.2, or 16.76±0.2.

6. The cocrystal according to claim 1, having an XRPD pattern, obtained with CuKα1-radiation, substantially as shown in FIG. 1.

7. The cocrystal according to claim 1, wherein the DSC curve of the cocrystal comprises an endotherm between about 143° C. and about 160° C.

8. The cocrystal according to claim 1, wherein the crystallinity is greater than 99%.

9. A pharmaceutical composition comprising a therapeutically effective amount of the cocrystal of claim 1, together with one or more pharmaceutically acceptable excipients.

10. The pharmaceutical composition according to claim 9, wherein the polymorphic purity of the cocrystal is at least about 99%.

11.-15. (canceled)

16. The cocrystal according to claim 7, wherein the DSC curve of the cocrystal comprises an endotherm at approximately 145.7° C.

17. A method for treating a disease or disorder in a subject, the method comprising administering to the subject a therapeutically effective amount of the cocrystal of claim 1,
wherein the disease or disorder is selected from the group consisting of: a cardiovascular disease; a disorder of fatty acid metabolism; a glucose utilization disorder; a gastrointestinal disease or disorder; a liver disease or disorder; a hyperabsorption syndrome; hypervitaminosis and osteopetrosis; hypertension; glomerular hyperfiltration; or pruritus of renal failure.

18. The method of claim 17, wherein the disease or disorder is selected from the group consisting of: a cardiovascular disease, a disorder of fatty acid metabolism, and a glucose utilization disorder.

19. The method of claim 18, wherein the cardiovascular disease, disorder of fatty acid metabolism, or glucose utilization disorder is selected from the group consisting of:
hypercholesterolemia; type 1 or type 2 diabetes mellitus; a complication of diabetes; and a diabetes-related disease.

20. The method of claim 19, wherein the diabetes-related disease is selected from the group consisting of: insulin resistance, hyperglycemia, hyperinsulinemia, elevated blood levels of fatty acids or glycerol, obesity, dyslipidemia, hyperlipidemia, metabolic syndrome, atherosclerosis, and hypertension.

21. The method of claim 17, wherein the disease or disorder is a gastrointestinal disease or disorder.

22. The method of claim 21, wherein the gastrointestinal disease or disorder is selected from the group consisting of: constipation; Crohn's disease; primary bile acid malabsorption; irritable bowel syndrome (IBS); inflammatory bowel disease (IBD); ileal inflammation; and reflux disease and complications thereof.

23. The method of claim 17, wherein the disease or disorder is a liver disease or disorder.

24. The method of claim 23, wherein the liver disease or disorder is selected from the group consisting of: inherited metabolic disorder of the liver; an inborn error of bile acid synthesis; a congenital bile duct anomaly; biliary atresia; post-Kasai biliary atresia; post-liver transplantation biliary atresia; neonatal hepatitis; neonatal cholestasis; hereditary forms of cholestasis; cerebrotendinous xanthomatosis; a secondary defect of BA synthesis; Zellweger's syndrome; cystic fibrosis-associated liver disease; alpha1-antitrypsin deficiency; Alagille syndrome (ALGS); Byler syndrome; a primary defect of bile acid (BA) synthesis; progressive familial intrahepatic cholestasis (PFIC); benign recurrent intrahepatic cholestasis (BRIC); autoimmune hepatitis; primary biliary cirrhosis (PBC); liver fibrosis; non-alcoholic fatty liver disease (NAFLD); non-alcoholic steatohepatitis (NASH); portal hypertension; cholestasis; Down syndrome cholestasis; drug-induced cholestasis; intrahepatic cholestasis of pregnancy (jaundice during pregnancy); intrahepatic cholestasis; extrahepatic cholestasis; parenteral nutrition associated cholestasis (PNAC); low phospholipid-associated cholestasis; lymphedema cholestasis syndrome 1 (LCS1); primary sclerosing cholangitis (PSC); immunoglobulin G4 associated cholangitis; primary biliary cholangitis; cholelithiasis (gallstones); biliary lithiasis; choledocholithiasis; gallstone pancreatitis; Caroli disease; malignancy of bile ducts; malignancy causing obstruction of the biliary tree; biliary strictures; AIDS cholangiopathy; ischemic cholangiopathy; pruritus due to cholestasis or jaundice; pancreatitis; chronic autoimmune liver disease leading to progressive cholestasis; hepatic steatosis; alcoholic hepatitis; acute fatty liver; fatty liver of pregnancy; drug-induced hepatitis; iron overload disorders; congenital bile acid synthesis defect type 1 (BAS defect type 1); drug-induced liver injury (DILI); hepatic fibrosis; congenital hepatic fibrosis; hepatic cirrhosis; Langerhans cell histiocytosis (LCH); neonatal ichthyosis sclerosing cholangitis (NISCH); erythropoietic protoporphyria (EPP); idiopathic adulthood ductopenia (IAD); idiopathic neonatal hepatitis (INH); non syndromic paucity of interlobular bile ducts (NS PILBD); North American Indian childhood cirrhosis (NAIC); hepatic sarcoidosis; amyloidosis; necrotizing enterocolitis; serum bile acid-caused toxicities; viral hepatitis; hepatocellular carcinoma (hepatoma); cholangiocarcinoma; a bile acid-related gastrointestinal cancer; and cholestasis caused by tumors and neoplasms of the liver, of the biliary tract and of the pancreas.

25. The method according to claim 17, wherein the liver disease or disorder is selected from the group consisting of: progressive familial intrahepatic cholestasis (PFIC), Alagilles syndrome, biliary atresia, and non-alcoholic steatohepatitis (NASH).

26. The method according to claim 17, wherein the hyperabsorption syndrome is selected from abetalipoproteinemia, familial hypobetalipoproteinemia (FHBL), chylomicron retention disease (CRD), and sitosterolemia.
